(19) United States
(12) Patent Application Publication
(10) Pub. No.: US 2006/0084615 A1
(43) Pub. Date: Apr. 20, 2006

(54) Puerarin derivatives and their medical uses

(76) Inventors: Albert Sun-Chi Chan, Kowloon (HK); Shi Lin Chen, Shenzhen City (CN); Yueming Li, Kowloon (HK); Dajian Yang, Shenzhen City (CN)

Correspondence Address: KNOBBE MARTENS OLSON & BEAR LLP, 2040 MAIN STREET, FOURTEENTH FLOOR, IRVINE, CA, 92614, US

(21) Appl. No.: 10/969,571

(22) Filed: Oct. 20, 2004

(51) Int. Cl. A61K 31/7048; C07H 17/00

(52) U.S. Cl. 514/27; 536/8

(57) ABSTRACT

The present invention provides acetylated derivatives of the compound puerarin that have enhanced bioavailability and are particularly suitable for oral administration. The present invention also teaches the use of medicaments containing acetylated derivatives of puerarin that are suitable for the treatment of myocardial ischemia and for modulating blood lipid levels, dilating coronary and cerebral arteries, reducing oxygen consumption of cardiomyocytes, improving microcirculation and preventing aggregation of blood platelets.

### FIELD OF INVENTION

The present invention is related to puerarin derivatives with enhanced bioavailability and methods of producing the same.

### BACKGROUND OF INVENTION

Puerarin is an active component isolated from the plant Pueraria lobata. It has been known for being effective in modulating blood lipid levels, dilating coronary and cerebral arteries, reducing oxygen consumption of cardiomyocytes, improving micro-circulation and has the function of preventing aggregation of blood platelets.

Puerarin has the general formula as shown in FIG. 1 where all the groups R1, R2 and R3 are hydrogen atoms. The low bioavailability is mainly attributed to the glucose group, which results in the low solubility of puerarin in lipids, thus rending puerarin not easily absorbed by the body.

As a result, the above-mentioned health benefits of puerarin are limited due to its relatively low bioavailability. Also because of this, the clinical route of administration of puerarin is limited to injections and the scope of application for puerarin is thus small.

It is an object of the present invention to provide modified forms of puerarin with enhanced bioavailability.

### SUMMARY OF THE INVENTION

One aspect of the present invention is to provide puerarin, a compound having the following formula as given in FIG. 1 wherein R1 is an acyl group of 2-5 carbon atoms; R2 is selected from a group consisting of hydrogen and an acyl group, said acyl group containing 2-5 carbon atoms; R3 is hydrogen.

Another aspect of the present invention is to provide derivatives of puerarin where either R1 or R2, or both, are acetyl groups.

A third aspect of the present invention is to provide tetra-acetylated puerarin (4ac) which is given by the structure:

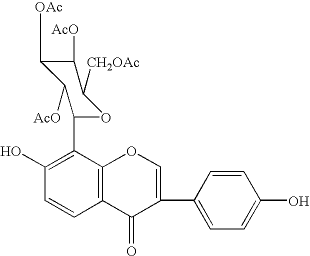

Another aspect of the present invention is a process of acetylating puerarin to produce a mixture comprising tetra-, penta-, and hexa-acetylated puerarin; removing materials that are soluble in an organic solvent from the mixture; and separating the tetra-, penta-, and hexa-acetylated puerarin by column chromatography.

Yet another aspect of the present invention is to provide an acetylated derivative of puerarin to modulate blood lipid levels, dilate coronary and cerebral arteries, reduce oxygen consumption of cardiomyocytes, improve microcirculation, prevent aggregation of blood platelets and treat myocardial ischemia.

Accordingly, another aspect of the present invention is a method of treatment for these diseases, the method comprising administering a pharmaceutically acceptable dose of any one of the compounds of puerarin or puerarin derivatives, or a combination of these compounds.

### BRIEF DESCRIPTION OF THE DRAWINGS

FIG. 1 shows the general structure of puerarin.

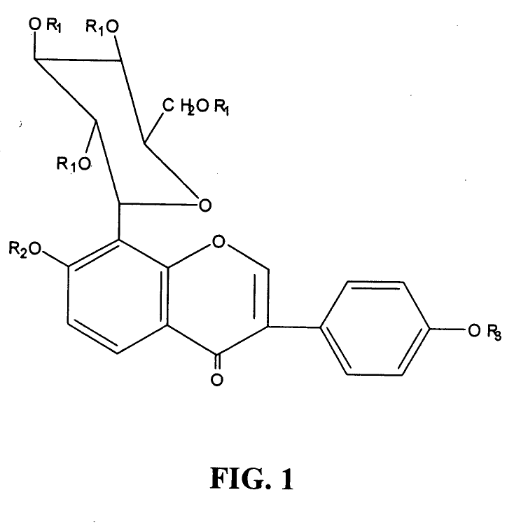

FIG. 2 is a structural representation of 2″,3″,4″,6″-0-tetraacetylpuerarin (4ac).

FIG. 3 shows the structural representation of 7,2″,3″,4″,6″-0-pentaacetylpuerarin.

FIG. 4 is the elution profile of the puerarin standard solution in serum after treatment using high performance liquid chromatography.

FIG. 5 is the elution profile of the puerarin standard solution in blank serum after treatment using high performance liquid chromatography.

FIG. 6 is the elution profile of the puerarin-containing serum after treatment using high performance liquid chromatography.

### DETAILED DESCRIPTION

The present invention includes the synthesis of compounds I-III and their structural identification in the following examples. Their respective bioavailability and efficacy are also illustrated.

### EXAMPLE 1

### The Synthesis of Compounds I-III and Structural Identification

1.1 The synthesis of compound I-III includes the following procedures:
(1) Fifty grams of puerarin (99% pure; Sha'anxi Huike Botanical Technology Co. Ltd., PRC) was dissolved in 600 ml pyridine and 100 g of acetic anhydride was added to the mixture. The mixture was then stirred at room temperature for 30 minutes and maintained at room temperature for another 24 hours. The resultant mixture is Reactant A.
(2) Reactant A was slowly poured into 10 L ice water. After sufficient stirring, the mixture was filtered at reduced pressure and approximately 80 g of Reactant B was obtained.
(3) Reactant B was dissolved in dichloromethane and 5% sodium carbonate solution was added. After stirring at room temperature for 1 hour, the organic phase was separated and allowed to evaporate to obtain 70 g of dry Reactant C.
(4) Reactant C was applied to a chromatographic column containing 5000 g of colloidal silica H (10-40μ, Qingdao Haiyang Chemicals Production Plant). The eluate was a mixture of petroleum ester and ethyl acetate, sequentially applied to the column in the ratios of 9:1, 5:1 and 3:2. The effluent was collected in different fractions. By inspection under thin-layer chromatography (TLC), the groups of chemicals with the same Retention Factor (R_{f}) values were combined. The solvent mixture was evaporated and the chemicals re-crystallized with acetone to produce approximately 15 g, 20 g and 25 g of compounds I, II and III respectively.

A person skilled in the art will recognize that alternatively, an equivalent molar amount of another suitable acetylating agent such as acetyl chloride instead of acetic anhydride may be used. Also, while dichloromethane was the organic solvent used, another suitable organic solvent may also be substituted in the above method.

1.2 Properties of Compounds I-III

(1) Compound I: 2″,3″,4″,6″-0-tetraacetylpuerarin

Melting point: 144-5° C.

[α]D20=+37.7 (c=2.2, CHCl3)

1H NMR (CDCl3, δ, ppm): 8.21 (s, 1H), 8.19 (d, 1H, J=9 Hz), 7.96 (s, 1H), 7.39 (d, 2H, J=8.5 Hz), 7.01 (d, 1H, J=9 Hz), 6.88 (d, 2H, J=8.5 Hz), 5.76 (s, br, 1H), 5.43 (m, 3H), 5.34 (m, 1H), 4.36 (dd, 1H, J1=3.5 Hz, J2=12 Hz), 4.21 (dd, 1H, J1=2.5 Hz, J2=13 Hz), 3.99 (m, 1H), 2.14 (s, 3H), 2.09 (s, 3H), 2.03 (s, 3H), 1.69 (s, 3H).

13C NMR (CDCl3, δ, ppm) 176.6, 170.9, 170.6, 169.8, 161.4, 156.8, 152.1, 130.5, 128.8, 125.1, 122.9, 117.4, 115.9, 108.2, 76.6, 73.9, 70.2, 68.1, 61.8, 20.8, 20.3

MS: 585.1808 (100%), 413.3010, 393.3213, 360.3675, 264.8656.

HRMS Found for C29H28O13+H, 585.1616, calculated: 585.1608. Found for C29H28O13+Na, 607.1408, calculated: 607.1428.

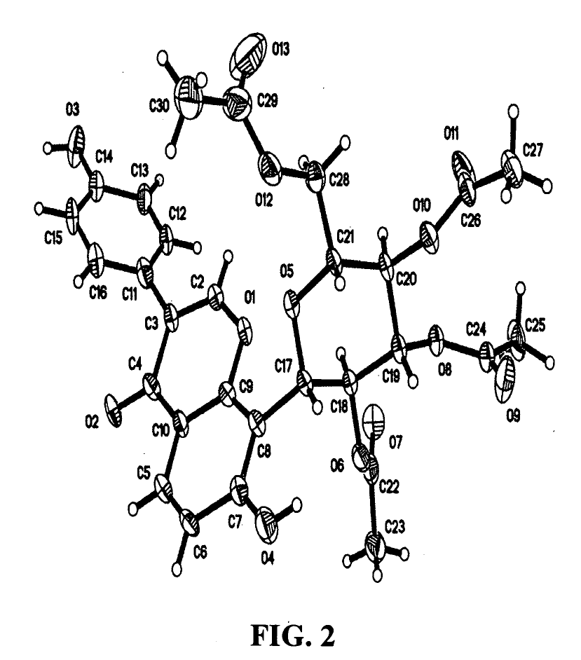

The colored needles were crystallized from acetone/hexane and were subjected to X-ray diffraction analysis. The structure 2″,3″,4″,6″-0-tetraacetylpuerarin is given in FIG. 2.

(2) Compound-II: 7,2″,3″,4″,1″-0-pentaacetylpuerarin

Melting point: 105-6° C.

[α]_{D}²⁰=+49.3 (c=2.0, CHCl₃)

¹H NMR (CDCl₃, δ, ppm) 8.20 (d, 1H, J=9.5 Hz), 8.0 (s, 1H), 7.57 (d, 2H, J=8.5 Hz), 7.17 (d, 2H, 8.5 Hz), 7.01 (d, 1H, 8.5 Hz), 5.40 (m, 3H), 5.35 (m, 1H), 4.37 (dd, 1H, J₁=3.5 Hz, J₂=12.5 Hz), 4.21 (dd, 1H, J₁=2 Hz, J₂=12.5 Hz), 3.99 (m, 1H), 2.32 (s, 3H), 2.14 (s, 3H), 2.09 (s, 3H), 2.02 (s, 3H), 1.67 (s, 3H).

¹³C NMR (CDCl₃, δ, ppm) 175.8, 171.0, 170.6, 169.8, 169.1, 161.5, 152.5, 150.9, 130.3, 129.3, 128.9, 117.7, 108.6, 74.0, 70.3, 68.3, 62.0, 21.4, 20.9, 20.8, 20.4

MS: 627.1688 (100%), 556.2772, 425.1861, 397.1928, 360.3266.

HRMS: Found for C₃₁H₃₀O₁₄+H, 627.1688, calculated: 627.1714. Found for C₃₁H₃₀O₁₄+Na: 649.1506, calculated: 649.1533.

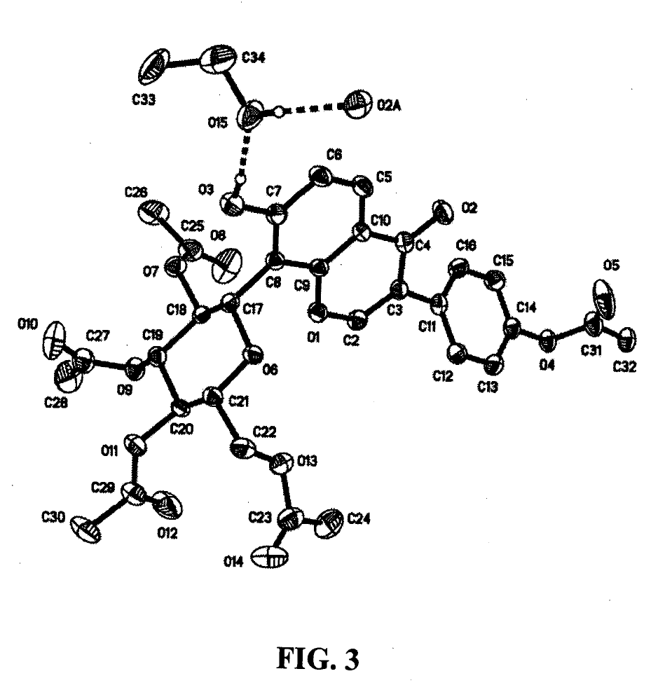

Colorless flakes were obtained from ethanol and subjected to X-ray diffraction analysis. The structure of 7,2″,3″,4″,6″-0-pentaacetylpuerarin is shown in FIG. 3.

(3) Compound III: Hexa-0-acetyl puerarin

Melting point: 118-9° C.

[α]D20=−45.5 (c=2.1, CHCl₃)

1H NMR (CDCl3, δ, ppm) 8.34 (d, 1H, J=9 Hz), 8.09 (s, 1H, br), 7.60 (d, 2H, J=8 Hz), 7.23 (m, 4H), 5.87-7.72 (1H, br), 5.43-5.28 (m, 2H), 3.88 (m, 1H), 2.45 (s, 3H), 2.33 (s, 3H), 2.08 (s, 6H), 2.03 (s, 3H), 1.70 (s, br, 3H).

MS: 669.1808 (100%), 556.2771, 481.1439, 413.2685, 360.3273, 297.6115.

HRMS: Found for C33H32O15+H, 669.1801, calculated: 669.1819. Found C33H32O15+Na: 691.1622, calculated: 691.1639.

Compounds I, II and III were identified to be the puerarin derivatives tetra-, penta- and hexa-acetylated puerarin respectively.

### EXAMPLE 2

### The Study of the Bioavailability of Orally-Administered Puerarin Derivatives

2.1 Materials

2.1.1 Drug and Reagents

Puerarin was bought from Beijing Union Pharmaceutical Factory (People's Republic of China, PRC; batch no: 030404). The purity was shown to be above 99% by high performance liquid chromatography (HPLC) analysis. The puerarin derivatives 4ac, 5ac and 6ac were synthesized as described and provided by The Hong Kong Polytechnic University. Their purities were above 98% as tested by that institute. Acetonitrile and methanol were of HPLC grade and the double distilled water was used.

2.1.2 Instrument and Chromatography Conditions

Agilent 1100 HPLC, DAD diode, HP1100 chromatography workstation and Agilent XDB-C18 column (250 mm×4.6 mm D, 5 μm) were used. The pre-column is Agilent XDB-C18 column (12.5 mm×4.6 mm D, 5 μm). Gradient elution was carried according to Table 1 below:

TABLE 1
Chromatography conditions

Ratio

| Time (min) | Acetonitrile | Water |
|---|---|---|
| 0 | 10 | 90 |
| 15 | 60 | 40 |
| 20 | 70 | 30 |
| 30 | 100 | 0 |

The flow rate was kept at 0.7 mL/min throughout the course. The column temperature was at room temperature and the detection was done at 250 nm. Other instruments included a Rotofix-32 bench top centrifuge (Hettich, Germany), an MS2 stirrer (Guangzhou Scientific Instrument Technology Co. Ltd., PRC) and a 5415D centrifuge (Eppendorf, Germany)

2.1.3 Animals

Sprague-Dawley (SD) rats weighing 180±20 g, both male and female were provided by the Laboratory Animal Centre of the Guangzhou Traditional Chinese Medicine (TCM) University.

2.2 Methods and Results

2.2.1 Collection of Samples

Two hundred and sixty SD rats were randomly divided into 20 groups with 13 rats in each group. The rats were fasted for one day before the test.

Puerarin, 4ac, 5ac and 6ac was dissolved in sterilized water to produce suspension of final dosage of 400 mg/kg, 560 mg/kg, 600 mg/kg and 640 mg/kg, 10 ml/kg and administered orally to the rats.

At t=10, 20, 30, 45, 60, 90, 120 min, 2 h, 4 h, 6 h, 7 h, 8 h and 12 h after administration, 3 ml of blood was collected from the femoral vein of each animal and kept in a centrifuge tube at room temperature for 0.5 h. The blood was centrifuged at 3000 r/min for 15 min and the serum was taken for analysis.

2.2.2 Treatment of Samples

Serum (0.5 ml) was added with 0.2 ml methanol and mixed with a vortex mixer for 1 min. The mixture was then centrifuged at 3000 r/min for 15 min. The supernatant was dried by nitrogen gas flow and the residue was dissolved in 0.2 ml methanol. The mixture was again centrifuged at 10000 r/min for 10 min and the supernatant subjected to HPLC analysis.

2.2.3 The Elution Profiles

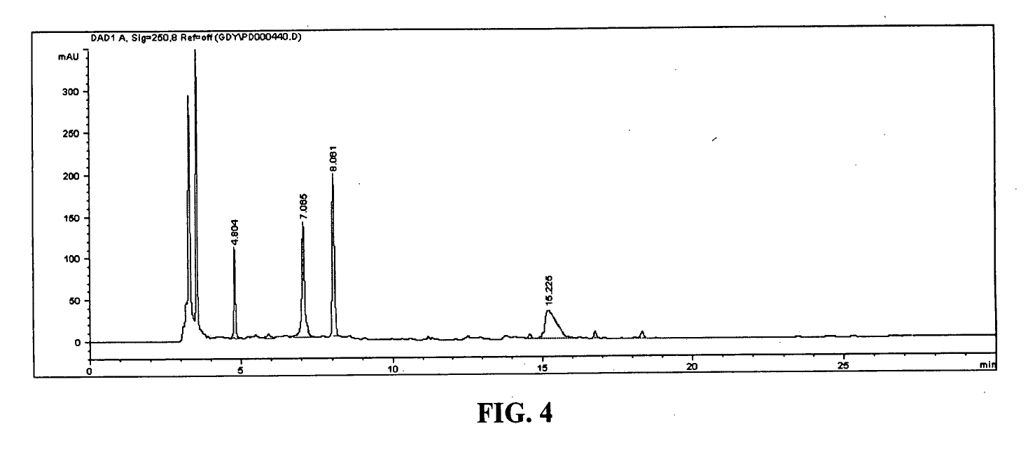

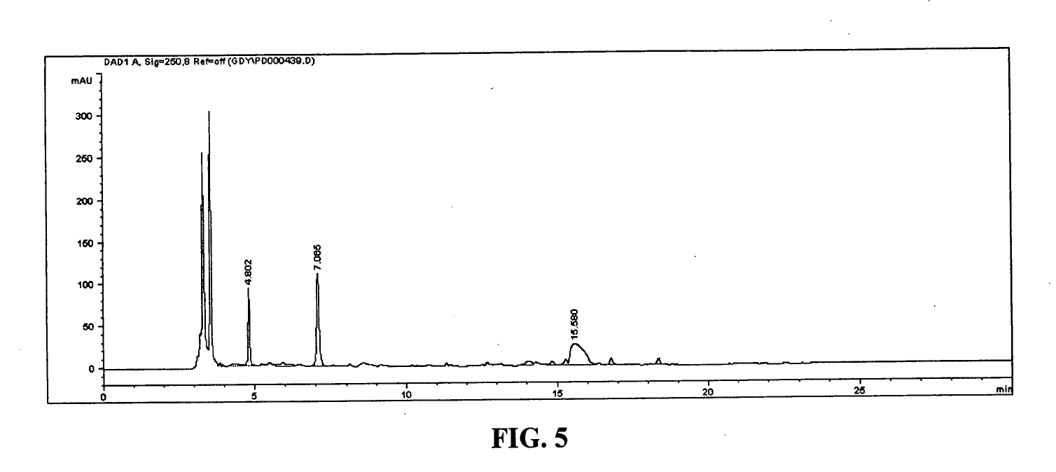

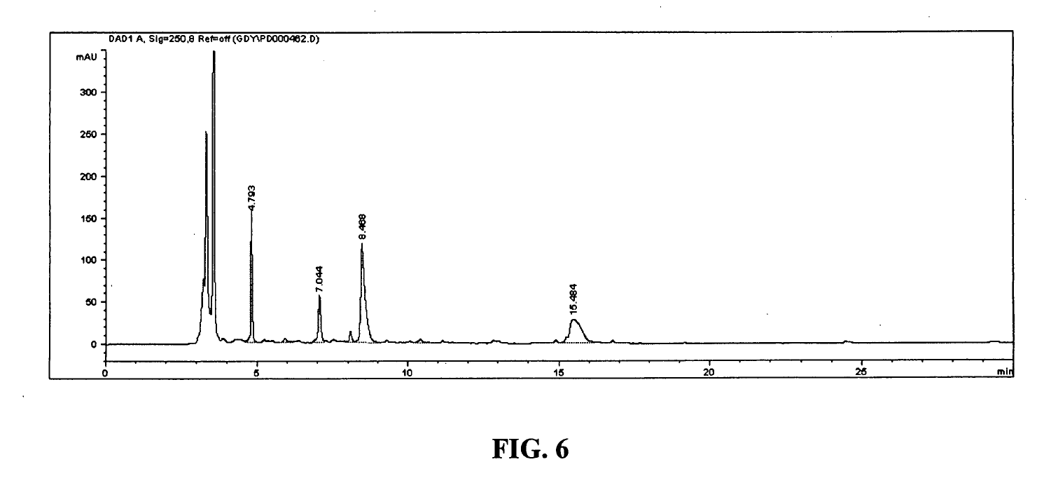

FIGS. 4, 5 and 6 are respectively the spectra of the puerarin standard solution in serum, blank serum and the puerarin-containing serum after treatment. The spectra showed that any inherent impurity in serum did not interfere with the detection of puerarin. The detention time of puerarin was 8.5 min.

2.2.4 Determination of Standard Curve

a) Standard solutions Standard solutions of puerarin at concentrations of 0.025, 0.0125, 0.00625, 0.003125 and 0.00156 mg/ml were prepared from the stock solution of 0.1 mg/ml of puerarin in methanol.

b) Chromatography protocol: 0.5 ml of each standard concentration was mixed with 0.5 ml blank serum and 20 μl of each of these solutions were subjected to HPLC analysis to detect their respective peaks.

c) Calibration curve: Using the concentration of puerarin as the abscissa (X-axis) and the peak area as the ordinate (Y-axis), the standard curves of the puerarin derivatives were obtained and shown as Chart 1.

d) Calculation of unknown concentrations of puerarin: The regression equation was Y=33426X+32.108 (R2=0.9996) as shown as Chart 1. The linearity was good in the range of 1.95 μg to 31.25 μg. The minimum amount detected is 195 ng the minimum concentration detected is 195 ng/ml. In the regression equation formula, b/a>100, so the one-point external standard method was used.

[image]

2.2.5 The Recovery Test

Six portions of 0.5 ml blank serum were taken and added with different volumes of the puerarin standard solution. They were mixed with a vortex mixer and 2.0 ml of methanol was added and further mixed for 1 min. The mixture was then centrifuged at 3000 r/min for 15 min. The supernatant was dried with nitrogen gas flow. The residues were dissolved in methanol and centrifuged at 10000 r/min for 10 min. The supernatants were then taken for HPLC analysis. The peak areas were recorded and the recovery percentages were obtained. The average recovery percentage was 92.03% for the low, medium and high concentrations as shown in Table 2.

TABLE 2
Recycling percentage test (n = 6)

| | | | | | Relative |
|---|---|---|---|---|---|
| | Amount | Amount | Recycling | | Standard |
| | added | detected | percentage | Mean | Deviation |
| No. | (Peak area) | (Peak area) | (%) | (%) | (RSD) (%) |
| 1 | 710.6 | 636.6 | 89.59 | | |
| 2 | 695.1 | 645.7 | 92.89 | | |
| 3 | 346.0 | 318.8 | 92.14 | | |
| 4 | 355.6 | 328.1 | 92.27 | 92.03 | 1.40 |
| 5 | 185.2 | 172.7 | 93.25 | | |
| 6 | 184.6 | 169.9 | 92.04 | | |

The results showed that the recovery percentages were acceptable and therefore this method was suitable and feasible.

2.2.6 Precision Test for the Methodology Used

Serum samples of 0.5 ml volume containing 0.00625 mg/ml (within-day precision test) and 0.003125 mg/ml (between-day precision test) puerarin were each mixed with 2.0 ml of methanol. The precision test was only carried out for puerarin and not for the derivatives because the derivatives will be converted in the body to puerarin. The mixtures were vigorously mixed for 1 min with a vortex mixture and centrifuged for 15 min at 3000 r/min. The supernatants were then dried by nitrogen gas flow. The residues were each dissolved in 0.2 ml of methanol. The mixtures were then further centrifuged at 10000 r/min for 10 min. The supernatants obtained were subjected to HPLC analysis. One-point external standard method was used to calculate the relative standard deviation (RSD; %). The results are shown in Tables 3 and 4.

TABLE 3
Within-day precision test (n = 5)

| | No. | | | | | | |
|---|---|---|---|---|---|---|---|
| | 1 | 2 | 3 | 4 | 5 | Mean | RSD |
| Peak area | 318.8 | 328.1 | 348.1 | 348.4 | 361.7 | 341.0 | 5.06% |

TABLE 4
Between-day precision test (n = 5)

| | No. | | | | | | |
|---|---|---|---|---|---|---|---|
| | 1 | 2 | 3 | 4 | 5 | Mean | RSD |
| Peak area | 645.7 | 636.6 | 610.5 | 542.8 | 562.1 | 599.54 | 7.57% |

The results showed that the relative standard deviations of the within-day and between-day precision test were less than 10%, and thus this method was of high precision.

2.2.7 Data Analysis

The samples were treated, applied and as stated in section 2.2, and the serum concentration of each compound was calculated by the one-point external standard method. The calculated data were processed using the 3P97 pharmacokinetics statistics software supplied by the Mathematics Committee of the China Pharmacology Association. The results showed that the in vivo metabolizing of puerarin, 5ac and 6ac fit into the two-compartment open model (Reid J M et al, Single-dose pharmacokinetics of the DNA-binding bioreductive agent NLCQ-1 (NSC 709257) in CD2F1 mice. Cancer Chemother Pharmacol. 51(6):483-7. 2003), while that of 4ac fits into the one-compartment open model (Egorin M J et al., Pharmacokinetics, tissue distribution, and metabolism of 17-(dimethylaminoethylamino)-17-demethoxygeldanamycin (NSC 707545) in CD2F1 mice and Fischer 344 rats. Cancer Chemother Pharmacol. 49(1): 7-19. 2002). This implies that 4ac equilibrates rapidly between the blood and the extravascular tissues and is suitable for oral administration. The major pharmacokinetic parameters are shown in Table 5 below.

TABLE 5
Pharmacokinetic parameters of puerarin and its derivatives

| Parameter | t_{1/2(a)} | t_{1/2(β)} | CL | AUC(0-∞) | T(peak) | C (max) |
|---|---|---|---|---|---|---|
| Sample | min | min | mg/kg/min/μg/ml | (μg/ml)min | min | μg/ml |
| Puerarin | 53.4221 ± 11.3186 | 60.9209 ± 0.4314 | 0.2032 ± 0.0541 | 2729.94 ± 491.99 | 84.91 ± 7.69 | 9.2253 ± 2.3726 |
| 4ac | | | 0.1030 ± 0.0163 | 6104.81 ± 275.29 | 51.48 ± 13.03 | 17.6146 ± 2.0624 |
| 5ac | 22.1168 ± 18.7136 | 425.03 ± 253.68 | 0.1011 ± 0.0425 | 4566.95 ± 762.64 | 42.23 ± 18.07 | 15.0431 ± 1.8110 |
| 6ac | 111.9702 ± 108.66 | 2664.97 ± 379.94 | 0.1696 ± 0.1206 | 3149.69 ± 467.26 | 81.39 ± 10.02 | 4.9324 ± 0.0695 |

t_{1/2(a)} = half-life of absorption
t_{1/2(β)} = half-life of clearance
CL = total body clearance
AUC = area under concentration-time curve
T (peak) = time the compound takes to reach the absorption peak
C (max) = maximum concentration reached by the compound

The area under the curve or AUC is the basic parameter used to calculate the absolute and relative bioavailability. It represents the extent of absorption of the tested compound: the larger the AUC, the higher the absorption.

In this experiment, the AUC of 4ac was the highest among the tested compounds. It was 2.24 folds of that of puerarin, demonstrating that it has better absorption and higher bioavailability than puerarin or other derivatives.

T(peak) reflects the rate of absorption of the compound; the higher the rate, the shorter the time to reach its maximum concentration C(max). This is an important evaluation parameter for a drug for which the time of onset needs to be very short. In this study, the T(peak) of 4ac, though not the smallest, was much smaller than that of puerarin, showing that the absorption rate of 4ac was faster than that of puerarin.

C(max) indicates the maximum concentration obtained in the concentration-time curve. This parameter also reflects the bioavailability of the tested compound. An optimal C(max) should be greater than the minimum effective concentration and less than the minimum toxic concentration. In this experiment, the C(max) of 4ac was the highest; it was 1.90 folds that of puerarin. The absorption of 4ac was the best among the tested compounds.

The area under curve (AUC) of 4ac was higher than that of the other groups. By the use of t test, the AUC of ⁴ac was significantly greater than that of puerarin (p<0.01).

The data showed that the bioavailability of 4ac was higher than puerarin and other derivatives, and suggest that 4ac may be beneficial in oral formulations of puerarin. The data also suggest that bioavailability does not correspond to the degree of acetylation. As shown by our experiments, a higher degree of acetylation does not necessarily mean better bioavailability of puerarin.

### EXAMPLE 3

### The Effect of Puerarin and its Derivatives on Acute Myocardial Ischemia Induced by Posterior Pituitary Extract Injection in Rats

3.1 Materials

3.1.1 Testing Compound

Puerarin was purchased from Beijing Union Pharmaceutical Factory (PRC). Puerarin derivatives 4ac, 5ac and 6ac were supplied by The Hong Kong Polytechnic University. The puerarin was dissolved in 1:1 of PEG400 and sterilized distilled water to make 0.8 g/kg body weight, 10 ml/kg body weight solutions for i.g. feeding.

Puerarin derivative 4ac was dissolved in 1:1 of PEG400 and sterilized distilled water to make 1.12 g/kg, 10 ml/kg solution for intra-gastro (i.g.) feeding. Puerarin derivative 5ac was dissolved in 1:1 of PEG400 and sterilized distilled water to make 1.20 g/kg, 10 ml/kg solution for i.g. feeding. Puerarin derivative 6ac was dissolved in 1:1 of PEG400 and sterilized distilled water to make 1.28 g/kg, 10 ml/kg solution for ig feeding. The concentrations of each solution were selected such that 0.8 g/kg of equivalent puerarin will be administered to each animal.

3.1.2 Positive Control

Propranolol hydrochloride at 10 mg/tablet, was from the Shantou Jinshi Pharmaceuticals (PRC, batch no: 020802). Propranolol hydrochloride is a drug prescribed for myocardial ischemia. It is expected to decrease the T wave elevation in ECG during myocardial ischemia. The drug was dissolved in sterilized distilled water to make a 0.2 mg/ml, 10 ml/kg solution for i.g. feeding.

3.1.3 Instruments

The physiological recorder RM6240B (Chengdu Instrument Manufacturing Co. Ltd.) was used.

3.2 Animals

SD rats of specific pathogen free (SPF) grade weighing 200±20 g of both sexes were fed with rat chow supplied by the Laboratory Animal Centre of the Guangzhou TCM University. The animal quality certificate number is Yuejianzhengzi 2002A005 from the Laboratory Animal Monitoring Institute of Guangdong Science Committee. The rats were housed under normal conditions for one week and allowed to adapt to the environment.

3.3 Methods and Results

Sixty SPF grade SD rats weighing 200±20 g of both sexes were grouped evenly into six groups according to their weight and sex, and fed i.g. according to the following protocol:
a) Normal: distilled water 10 ml/kg;
b) Positive control: propranolol hydrochloride 0.2 tablet/ml, 10 ml/kg;
c) Puerarin solution: puerarin 0.8 g/kg, 10 ml/kg;
d) Puerarin derivative 4ac solution: 4ac 1.12 g/kg, 10 ml/kg;
e) Puerarin derivative 5ac solution: 5ac 1.20 g/kg, 10 ml/kg;
f) Puerarin derivative 6ac solution: 6ac 1.28 g/kg, 10 ml/kg.

The rats were administered the respective treatments for five consecutive days. On the sixth day, one hour after the administration of the treatment, the rats were anesthetized with 3% pentobarbital sodium at a dose of 40 mg/kg i.p. Two terminal leads were connected to the rats to record the normal electrocardiograph (ECG).

When the ECG reading became steady, 1 u/kg of posterior pituitary extract injection (obtained from the Nanjing Xintian Biochemical Pharmaceuticals Company Ltd; batch no 020601) was given intraveneously at the tail vein. Posterior pituitary extract induces acute myocardial ischemia. On ECG, the extract causes the T wave to elevate, flatten and sometimes invert. It also elevates the ST segment and lengthens the PR and QT intervals.

The ECG was recorded immediately for 30 minutes. The variations in T wave were calculated and analyzed statistically. The results are shown in Table 6 (on separate page).

Table 6 showed that the positive control, puerarin, 4ac, 5ac and 6ac groups showed significant differences in response to induced acute myocardial ischemia compared to the control group at t=15 s, 30 s, 2 min and 5 min.

This data suggest that puerarin and its derivatives 4ac, 5ac and 6ac are effective in ameliorating myocardial ischemia induced by posterior pituitary extract injection.

3.4 Conclusion

Puerarin and its derivatives 4ac, 5ac and 6ac were effective against myocardial ischemia in rats induced by posterior pituitary injection at doses of 0.8 g/kg, 1.12 g/kg, 1.20 g/kg and 1.28 g/kg (the latter three dosages were equivalent to 0.8 g/kg puerarin) respectively.

The acetylated derivatives of puerarin also had higher bioavailability than puerarin and exerted a better effect than puerarin alone. However, there is an optimal level of acetylation of puerarin as was determined by the inventor.

As derivatives of puerarin, acetylated derivatives of puerarin should also be efficacious in modulating blood lipid levels, dilating coronary and cerebral arteries, reducing oxygen consumption of cardiomyocytes, improving microcirculation and preventing aggregation of blood platelets.

It will be clear to a person skilled in the art that a composition containing puerarin or its derivatives, or a combination thereof, will be efficacious in the treatment of myocardial ischemia. A person skilled in the art will also recognize that while specific methods were taught, any number of variations and modifications may be made to the present invention while remaining within the scope and spirit of the present invention.

### REFERENCES FOR TECHNIQUES USED IN THE EXAMPLES

The references cited in this application are hereby incorporated by reference in their entirety.
a) Technical Requirement in Pharmacology and Toxicology Study of New Drug from TCM. State Drug Administration. (Examples 2 & 3)
b) Xu S Y et. Methodology of Pharmacological Experiments. Peoples' Hygiene Press, Beijing. 2002. (Example 3)
c) Chen Q. et. Methodology of TCM Pharmacology Study. Peoples' Hygiene Press, Beijing. 1993. (Example 3)

d) Wang B Y et. Technology and Methodology of Research and Development of New Drug from TCM. Shanghai Science and Technology Press, Shanghai. 2001. (Examples 2 & 3)

TABLE 6
The effect of puerarin and its derivatives on the T wave variations of
myocardial ischemic rats induced by posterior pituitary injection (X ± SD, n = 10).

| | Dosage/ | Amplitude of T wave (μV) | | | | | |
|---|---|---|---|---|---|---|---|
| Group | (g/kg⁻¹) | 5 s | 15 s | 30 s | 2 min | 5 min | 10 min |
| Control | | 15.69 ± 7.17 | 105.23 ± 33.09 | 66.25 ± 31.98 | 75.15 ± 27.29 | 70.62 ± 28.30 | 19.26 ± 10.11 |
| +ve | 2 tablets/kg | 16.11 ± 5.30 | 18.51 ± 9.26** | 22.10 ± 11.23** | 30.41 ± 11.58** | 26.62 ± 10.99** | 22.09 ± 11.31 |
| control | | | | | | | |
| Puerarin | 0.8 | 18.06 ± 10.09 | 25.69 ± 11.21** | 31.22 ± 14.59** | 39.20 ± 16.11** | 29.10 ± 16.23** | 25.01 ± 10.40 |
| 4ac | 1.12 | 15.09 ± 7.62 | 23.23 ± 10.69** | 35.40 ± 10.93* | 36.51 ± 16.02** | 25.60 ± 14.23** | 25.41 ± 10.21 |
| 5ac | 1.20 | 19.09 ± 6.20 | 29.42 ± 9.39** | 37.61 ± 13.43* | 40.11 ± 16.04** | 24.50 ± 13.63** | 24.39 ± 14.28 |
| 6ac | 1.28 | 16.33 ± 9.18 | 29.09 ± 16.50* | 39.12 ± 15.21* | 41.05 ± 15.60** | 29.14 ± 10.60** | 22.11 ± 9.81 |

**P < 0.01
*P < 0.05 compared to the control group

What is claimed is:

1. A compound having the following formula: 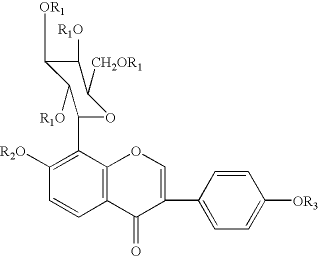
wherein R₁ is an acyl group of 2-5 carbon atoms; R₂ is selected from a group consisting of hydrogen and acyl group, said acyl group containing 2-5 carbon atoms; R₃ is hydrogen.

2. The compound according to claim 1, wherein R₁ is an acetyl group.

3. The compound according to claim 1, wherein R₂ is an acetyl group.

4. The compound according to claim 1, wherein both R₁ and R₂ are acetyl groups.

5. The compound according to claim 1, wherein the compound is tetra-acetylated puerarin (4ac) and where the structure is given as

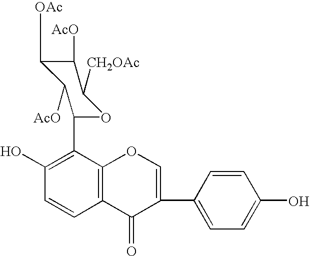

6. A pharmaceutical composition comprising a compound of the following formula: 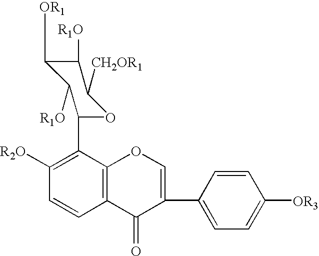
wherein R₁ is an acyl group of 2-5 carbon atoms; R₂ is selected from a group consisting of hydrogen and acyl group, said acyl group containing 2-5 carbon atoms; R₃ is hydrogen.

7. The pharmaceutical composition according to claim 6, wherein R₁ is an acetyl group.

8. The pharmaceutical composition according to claim 6, wherein R₂ is an acetyl group.

9. The pharmaceutical composition according to claim 6, wherein both R₁ and R₂ are acetyl groups.

10. The pharmaceutical composition according to claim 6, wherein the compound is tetra-acetylated puerarin (4ac) and where the structure is given as

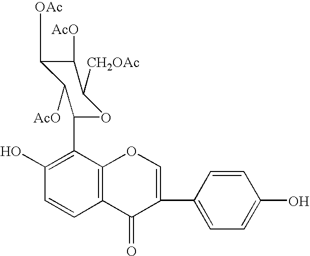

11. A pharmaceutical composition comprising a compound of the following formula: 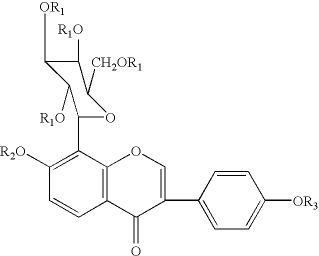
wherein R₁ is an acyl group of 2-5 carbon atoms; R₂ is selected from a group consisting of hydrogen and acyl group, said acyl group containing 2-5 carbon atoms; R₃ is hydrogen,
and wherein the pharmaceutical composition is for the treatment of myocardial ischemia.

12. The pharmaceutical composition according to claim 11, wherein R₁ is an acetyl group.

13. The pharmaceutical composition according to claim 11, wherein R₂ is an acetyl group.

14. The pharmaceutical composition according to claim 11, wherein both R₁ and R₂ are acetyl groups.

15. The pharmaceutical composition according to claim 11, wherein the compound is tetra-acetylated puerarin (4ac) and where the structure is given as

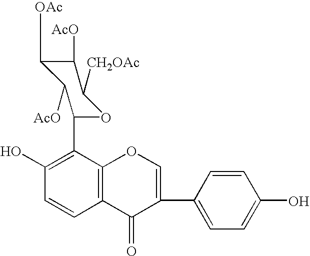

16. A process of producing the compound of claim 1, comprising
a) acetylating puerarin to produce a mixture comprising tetra-, penta-, and hexa-acetylated puerarin;
b) removing materials that are soluble in an organic solvent from the mixture; and
c) separating tetra-, penta-, and hexa-acetylated puerarin by column chromatography.

17. The process according to claim 16, wherein the acetylating in a) comprises acetylating puerarin with at least one acetylating agent.

18. The process according to claim 9, wherein the acetylating agent is selected from a group comprising acetic anhydride and acetyl chloride.

19. The process according to claim 8, wherein the organic solvent used is selected from a group comprising dichloromethane.

20. A method for enhancing the bioavailability of puerarin, wherein puerarin is given by the following formula: 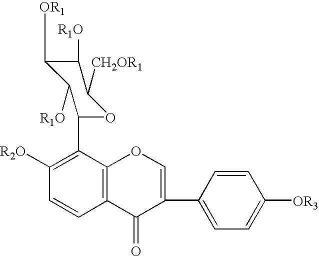
and the method comprising substituting R₁ with at least one acetyl group of 2-5 carbon atoms.

21. A method according to claim 12, the method further comprising substituting R₂ with at least one acetyl group of 2-5 carbon atoms.

22. A method according to claim 12, the method further comprising substituting R₂ with one hydrogen atom.

23. Method of treatment for a disease, the method comprising administering a pharmaceutically acceptable dose of a pharmaceutical composition having the following formula: 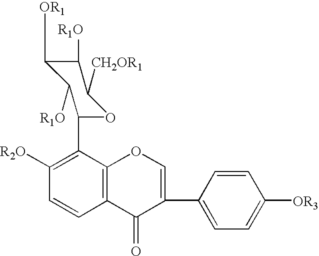
wherein R₁ is an acyl group of 2-5 carbon atoms; R₂ is selected from a group consisting of hydrogen and acyl group, said acyl group containing 2-5 carbon atoms; R₃ is hydrogen.

24. The method according to claim 23 wherein the disease is myocardial ischemia.

25. The method according to claim 23 wherein wherein R1 is an acetyl group.

26. The method according to claim 25 wherein the disease is myocardial ischemia.

27. The method according to claim 23 wherein R2 is an acetyl group.

28. The method according to claim 27 wherein the disease is myocardial ischemia.

29. The method according to claim 23 wherein both R1 and R2 are acetyl groups.

30. The method according to claim 29 wherein the disease is myocardial ischemia.

31. The method according to claim 23 wherein the compound is tetra-acetylated puerarin (4ac) and where the structure is given as

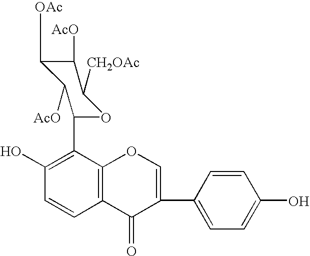

32. A method according to claim 31 wherein the disease is myocardial ischemia.
